(19) United States
(12) Patent Application Publication
(10) Pub. No.: US 2024/0156335 A1
(43) Pub. Date: May 16, 2024

(54) CONTINUOUS FLOW ENDOSCOPE

(71) Applicant: Covidien LP, Mansfield, MA (US)

(72) Inventors: Nikolai D. Begg, Wellesley, MA (US); Dalia Leibowitz, Cambridge, MA (US); Mireille Akilian, Candia, NH (US)

(21) Appl. No.: 18/407,277

(22) Filed: Jan. 8, 2024

Related U.S. Application Data
(63) Continuation of application No. 15/603,209, filed on May 23, 2017, now Pat. No. 11,864,735.
(60) Provisional application No. 62/341,908, filed on May 26, 2016.

(51) Int. Cl. A61B 1/015; A61B 1/00; A61B 1/018; A61B 1/313

(52) U.S. Cl. CPC A61B 1/015; A61B 1/00128; A61B 1/00165; A61B 1/018; A61B 1/3132; A61B 1/00167; A61B 1/07

(57) ABSTRACT

A continuous flow endoscope device includes an elongated tubular member defining an interior, a first channel disposed within the interior of the elongated tubular member, a second channel disposed within the interior of the elongated tubular member, and an optics device disposed in a free space of the interior of the elongated tubular member. The second channel is configured to receive an instrument therein during a first portion of an operation. A lumen defined through the second channel is fluidly isolated from a lumen defined through the first channel. The free space of the interior of the elongated tubular member being a space within the interior of the elongated tubular member not occupied by either the first channel or the second channel.

### CROSS-REFERENCE TO RELATED APPLICATIONS

This application is a continuation of U.S. patent application Ser. No. 15/603,209, filed on May 23, 2017, which claims the benefit of, and priority to, U.S. Provisional Patent Application No. 62/341,908, filed on May 26, 2016, the entire contents of each of which are hereby incorporated herein by reference.

### BACKGROUND

Various types of endoscopes may be employed for surgical and exploratory procedures, some of which may involve fluid inflow, outflow, or both depending upon the operation being performed. Depending upon the operation, there may be challenges to entry, access, or removal, or other challenges during and after the procedure, and the endoscope employed may need to adapt to these challenges by being configured for minimally invasive procedures or other procedures developed in response to those challenges.

### SUMMARY

As used herein, the term “distal” refers to the portion that is being described which is further from a user, while the term “proximal” refers to the portion that is being described which is closer to a user. Further, to the extent consistent, any of the aspects described herein may be used in conjunction with any or all of the other aspects described herein.

Provided in accordance with aspects of the present disclosure is a continuous flow endoscope device including an elongated tubular member, a first channel, a second channel, and an optics device. The elongated tubular member defines an interior. The first channel is disposed within the interior of the elongated tubular member. The second channel is disposed within the interior of the elongated tubular member and is configured to receive an instrument therein during a first portion of an operation. A lumen defined through the second channel is fluidly isolated from a lumen defined through the first channel. The optics device is disposed in a free space of the interior of the elongated tubular member. The free space is a space within the interior of the elongated tubular member not occupied by either the first channel or the second channel.

In an aspect of the present disclosure, the first channel is dedicated to fluid inflow and the second channel is dedicated to fluid outflow. Alternatively, the first channel may be dedicated to fluid outflow and the second channel dedicated to fluid inflow.

In another aspect of the present disclosure, the first channel defines a moon-shaped cross-sectional configuration. Additionally or alternatively, the second channel defines an elliptical cross-sectional configuration.

In yet another aspect of the present disclosure, the optics device is disposed within an oculiform region disposed within the interior of the elongated tubular member.

In still another aspect of the present disclosure, second channel is configured to operate without an instrument therein during a second portion of the operation. Further, the first and second channels may be configured to maintain continuous fluid flow during both the first and second portions of the operation.

In still yet another aspect of the present disclosure, the second channel is disposed within the first channel.

In an aspect of the present disclosure, the second channel is removable from the interior of the elongated tubular member.

Another continuous flow endoscope device provided in accordance with aspects of the present disclosure includes a first elongated tubular member defining an interior, a first channel disposed within the interior of the elongated tubular member, a second elongated tubular member defining a second channel therein, and an optics device. The second elongated tubular member is removably disposed within the interior of the first elongated tubular member. The second channel is configured to receive an instrument therein during a first portion of an operation. A lumen defined through the second channel is fluidly isolated from a lumen defined through the first channel. An optics device is disposed in a free space of the interior of the first elongated tubular member. The free space is a space within the interior of the first elongated tubular member not occupied by either the first channel or the second elongated tubular member.

In an aspect of the present disclosure, the first channel is dedicated to fluid inflow and the second channel is dedicated to fluid outflow. Alternatively, the first channel may be dedicated to fluid outflow and the second channel dedicated to fluid inflow.

In another aspect of the present disclosure, the first channel defines a moon-shaped cross-sectional configuration. Additionally or alternatively, the second channel defines an elliptical cross-sectional configuration.

In yet another aspect of the present disclosure, the optics device is disposed within an oculiform region disposed within the interior of the elongated tubular member.

In still another aspect of the present disclosure, second channel is configured to operate without an instrument therein during a second portion of the operation. Further, the first and second channels may be configured to maintain continuous fluid flow during both the first and second portions of the operation.

In still yet another aspect of the present disclosure, the second channel is disposed within the first channel.

In an aspect of the present disclosure, the second elongated tubular member is configured to releasably lock in engagement with the first elongated tubular member.

### BRIEF DESCRIPTION OF THE DRAWINGS

The above and other aspects and features of the present disclosure will become more apparent in view of the following detailed description when taken in conjunction with the accompanying drawings, wherein:

FIG. 1A is a perspective view of a surgical system provided in accordance with the present disclosure including a continuous flow endoscope device;

FIG. 1B is a transverse, cross-sectional view of the continuous flow endoscope of FIG. 1A;

FIG. 2 is a longitudinal, cross-sectional view of another continuous flow endoscope device provided in accordance with aspects of the present disclosure, disposed in an assembled condition;

FIG. 3 is a transverse, cross-sectional view of the continuous flow endoscope of FIG. 2, disposed in the assembled condition;

FIG. 4 is a longitudinal, cross-sectional view of the continuous flow endoscope of FIG. 2, disposed in a disassembled condition;

FIG. 5 is a transverse, cross-sectional view of the continuous flow endoscope device of FIG. 2, disposed in a disassembled condition; and

FIG. 6 is a schematic representation illustrating a fluid circuit of a continuous flow endoscope device modeled on a first-order basis as two resistors in series.

### DETAILED DESCRIPTION

Medical endoscopes often contain channels in addition to their optical components in order to allow access for surgical instruments and fluid flow in and out of the operative field. In procedures conducted within a liquid environment, such as hysteroscopy, blood or other operative debris may cause impaired visualization. Visualization in a bloody or debris-filled liquid may be improved and maintained with continuous flow, because the constant circulation of fluid through the operative field continuously decreases the concentration of blood and/or debris. Continuous flow is achieved by providing separate pathways for fluid inflow and outflow from the operative field. In some cases, the addition of multiple channels within the endoscope creates a challenge with respect to maintaining an outer diameter of the endoscope small enough to provide atraumatic access to the operative field.

Endoscopes may be configured with a single channel shared by inflow and instrument access, and a removable outflow cannula which is inserted when there is no instrument in the channel. However, this configuration is not able to provide true continuous flow throughout the procedure.

In order to provide continuous flow as well as instrument access, endoscopes may be configured in a number of ways. For example, an endoscope may be provided with three channels, one each for instrument access, inflow, and outflow. An endoscope may alternatively be provided, as another example, with two channels, one for inflow and instrument access, and the other for outflow. As another example, an endoscope may be provided with a detachable outer sheath for outflow, wherein an outflow channel is created by the annular space between the outer surface of the endoscope and the inner surface of the sheath. Such an endoscope may further contain either two individual channels for inflow and instrument access or one shared channel for inflow and instrument access. Rather than providing a separate outflow channel and shared inflow and instrument access channel, this configuration may be reversed. That is, a separate inflow channel may be provided while the shared channel is utilized for outflow and instrument access.

In endoscope configurations where a dedicated channel and a shared channel are provided, when there is no instrument within the shared channel, there is significantly more cross-sectional area for flow in the shared channel than in the dedicated channel. As a result, the fluid resistance through the shared channel will be significantly less than through the dedicated channel. Since fluid flow rate is directly proportional to pressure difference and indirectly proportional to fluid resistance, it will take significantly greater pressure difference across the dedicated channel to achieve the same fluid flow rate as through the shared channel. If the shared channel is used for fluid outflow during a procedure, fluid will flow out of the operative field with much less resistance than into the operative field, and it will likely be difficult to keep the operative field filled. This is especially true in procedures such as hysteroscopy where the operative field is pressurized to distend tissue and create space. The significant pressure difference across the outflow channel would make sufficient fluid inflow and pressure control within the cavity highly difficult to achieve.

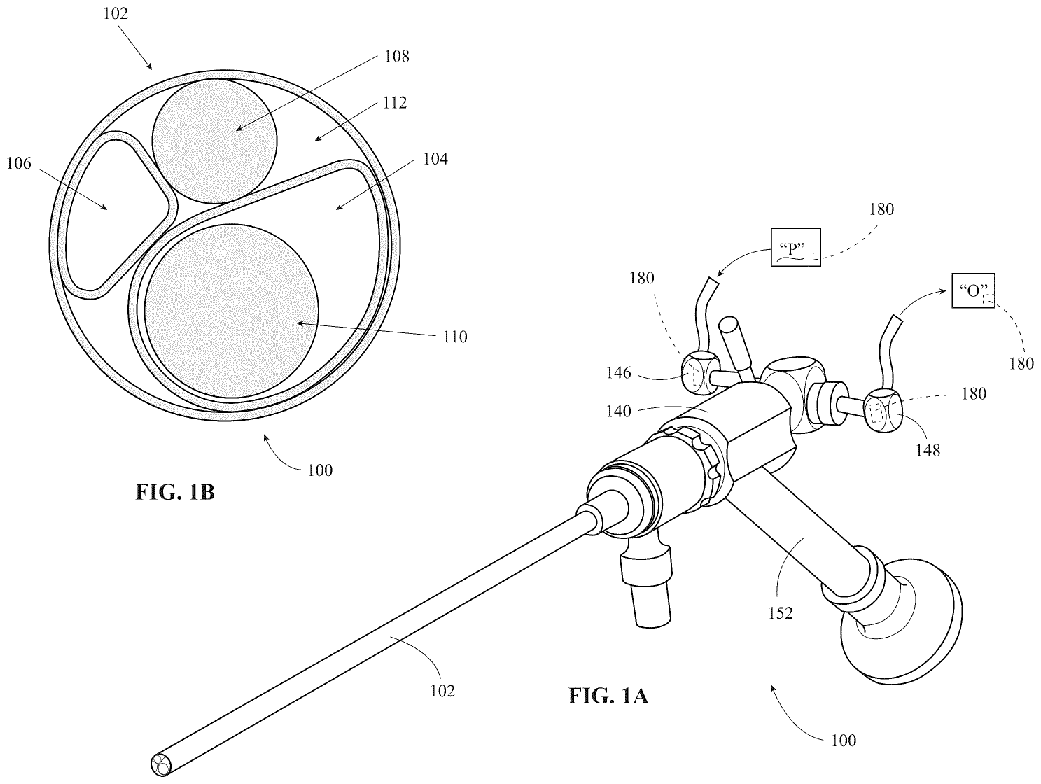

Referring generally to FIGS. 1A and 1B, FIG. 1A is a perspective view of a continuous flow endoscope device 100 provided in accordance with the present disclosure. Continuous flow endoscope device 100 includes an elongated tubular member 102 and a proximal body 140. Proximal body 140 includes an inflow 146, an outflow 148, and an arm 152 that is connected to an imaging device (e.g., a camera) to capture images received via a visualization device, e.g., optics 108 (FIG. 1B), extending through elongated tubular member 102. Continuous flow endoscope device 100 forms a system in conjunction with pump “P” in communication with inflow 146 and/or an outflow reservoir “0” in communication with outflow 148. The system may be configured as an open system, wherein pump “P” and outflow reservoir “0” are separate, or may be a closed or partially-closed system, wherein outflow reservoir “0” is coupled to pump “P” or incorporated therein.

FIG. 1B is a transverse, cross-sectional view of the elongated tubular member 102 of continuous flow endoscope device 100 (FIG. 1A) including an instrument 110, e.g., a morcellator or other suitable surgical instrument, inserted therethrough. In some embodiments, elongated tubular member 102 defines a first channel 104 that is shared between fluid flow and instrument access, e.g., for instrument 110, and a second channel 106 for fluid flow as well. In some embodiments, the first channel 104 is shared between the instrument 110 and fluid outflow and, thus, is coupled to outflow 148 (FIG. 1A), while the second channel 106 is employed for fluid inflow and, thus, is coupled to inflow 146 (FIG. 1A). In other embodiments, the first channel 104 is shared between the instrument 110 and fluid inflow (and, thus, is coupled to inflow 146 (FIG. 1A)), while the second channel 106 is employed for fluid outflow (and, thus, is coupled to outflow 148 (FIG. 1)).

Optics 108 extend through elongated tubular member 102 within a free space 112 thereof that is outside of the first and second channels 104, 106, respectively. This free space 112 may constitute any portion of the interior lumen defined by elongated tubular member 102 other than the portions occupied by the first and second channels 104, 106, respectively.

Referring still to FIGS. 1A and 1B, in embodiments of the present disclosure, the first and the second channels 104, 106, respectively, of the elongated tubular member 102 of continuous flow endoscope device 100 are configured both individually and relatively such that fluid resistance through the first channel 104 does not decrease to a level significantly lower than the fluid resistance through the second channel 106, regardless of whether an instrument 110 is inserted through first channel 104. “Significantly lower” and “significantly similar” as utilized herein may refer to when fluid flow through the first channel 104 is within a predetermined range of the fluid flow through the second channel 106. Other components of continuous flow endoscope 100 and/or the system including the same are additionally or alternatively configured to maintain a substantially similar resistance between the first and second channels 104, 106, respectively, thus keeping the fluid flow between the channels 104, 106 substantially similar (within a predetermined range of each other).

In some embodiments, the cross-sectional areas accessible by fluid flow in both the first and second channels 104, 106, respectively, are relatively similar (with or without an instrument 110 inserted through one of the channels 104, 106). As a result, the fluid resistances created by the configuration of the first and second channels 104, 106, respectively, are substantially similar. The elongated tubular member 102 of continuous flow endoscope device 100 may include a variety of channel configurations and profile shapes, which allows for true continuous flow with or without an instrument 110 placed in the first channel 104, regardless of whether the first channel 104 is used for fluid outflow or fluid inflow. The determination as to whether the first channel 104 is used for fluid inflow or fluid outflow may be based on the type of procedure being performed, the patient, the involved medical professionals, and other factors that may impact the type and size of instrument(s) 110 employed in the procedure so that when the instrument(s) 110 are removed/replaced, fluid resistance is maintained between the first and second channels 104, 106, respectively.

The first and second channels 104, 106, respectively, may be configured to be different sizes and/or shapes (geometries), and may be permanently fixed within elongated tubular member 102. The elongated tubular member 102 also accommodates the optics device 108 while true continuous fluid flow is occurring in the first and second channels 104, 106, respectively, regardless of whether an instrument 110 is present in the first channel 104 or the second channel 106. The first channel 104 and/or the second channel 106 may taper the diameter of their respective cross-sections along the length of the elongated tubular member 102 in the proximal-to-distal direction. The first and second channels 104, 106, respectively, are not in communication with each other, e.g., are separate from one another. Further, no sheath is required for use with continuous flow endoscope device 100.

The cross-section of the channel shared between fluid flow and instrument access, e.g., first channel 104, has an inner diameter greater than the outer diameter of instruments, e.g., instrument 110, inserted therethrough in order to enable flow in the resulting annular space. This shared channel 104 may also have a cross-section shaped differently from the outer profile of the instrument 110 in order to create additional space for fluid flow. As illustrated in FIG. 1B, the cross-section of the shared channel 104 may, more specifically, include a portion that complements the outer profile of the instrument 110 and another portion that does not, e.g., extends away from, the outer profile of the instrument 110 to create the additional space for fluid flow.

The first and second channels 104, 106, respectively, may define a cross-sectional geometry of a circle, polygon, polygon with rounded edges, kidney, bean, teardrop, half-moon, triangle, and combinations thereof. The first and second channels 104, 106, respectively, may define different cross-sectional geometries or similar geometries, or similar geometries with different relative scales. The channels 104, 106 may be formed from rigid or semi-rigid biocompatible material and, as noted above, are disposed within the elongated tubular member 102 of continuous flow endoscope device 100 without the aid of a sheath.

Instruments 110 used in conjunction with continuous flow endoscope device 100 may be relatively large and take up a significant portion of the cross-section of the lumen of elongated tubular member 102 of continuous flow endoscope device 100 when inserted through first channel 104. As a result, the first channel 104 may be relatively large and, together with the optics 108, may leave little space remaining for the second channel 106, which is, as a result, relatively small when compared with the first channel 104. When an instrument 110 is placed within the first channel 104, regardless of whether the first channel 104 is used for inflow or outflow, the cross-sectional areas accessible by fluid flow in both the first and second channels 104, 106 are substantially similar, so the fluid resistance of the first and second channels 104, 106 are similar, for example, when the length of the first channel 104 and the length of the second channel 106 are substantially similar (within a predetermined range of each other).

In some embodiments, the substantially similar resistance in the first and second channels 104, 106 may be achieved and/or maintained by an automatic, manual, electrical, mechanical, or electro-mechanical fluid control mechanism 180 of the pump “P” and/or the outflow reservoir “0.” Thus, the presence or absence of an instrument 110 within first channel 104 can be accounted for (or further accounted for).

In embodiments, the first channel 104 is used for fluid inflow and, thus, the fluid resistance of the second channel 106 (that has a smaller cross-section than the first channel 104) will be higher, allowing the operative field to remain pressurized. In embodiments where the first channel 104 is used for fluid inflow during a procedure, the fluid resistance of the fluid inflow will change throughout the procedure as instruments 110 are inserted into and removed from continuous flow endoscope device 100, since various instruments 110 may be employed throughout a procedure. In embodiments where continuous flow endoscope device 100 is used in combination with an external fluid control pump “P” (incorporating a fluid control mechanism 180 therein), the pump “P” will regulate the fluid inflow supply pressure in the first channel 104 in order to control the fluid pressure inside the operative space (e.g., the body cavity). In order to more accurately control pressure, the external fluid control pump “P” may employ the resistance of the fluid inflow path to calculate the theoretical pressure at the distal end of the elongated tubular member 102 of continuous flow endoscope device 100.

In some embodiments, the design of the channels 104, 106 may be sufficient to maintain the resistance of fluid flow. In some embodiments, if this measured resistance changes during the procedure and exceeds a predetermined amount, the fluid control mechanism 180 of the pump “P” is utilized to maintain the substantially similar resistances of fluid flow to control the pressure within the operative field to a desired accuracy. In some embodiments, the fluid control mechanism 180 of the pump “P” is not capable of maintaining such resistances. In these embodiments, an additional feature, such as another fluid control mechanism 180 associated with the outflow reservoir “0” coupled to the outflow 148, may be employed additionally or alternatively to maintain the fluid resistance of the fluid inflow path at a constant value.

In some embodiments, the first channel 104 is used for outflow, and the fluid resistance of the second (inflow) channel 106 does not change during the procedure, allowing the pump “P” to accurately control pressure because of the design of the respective channels 104, 106.

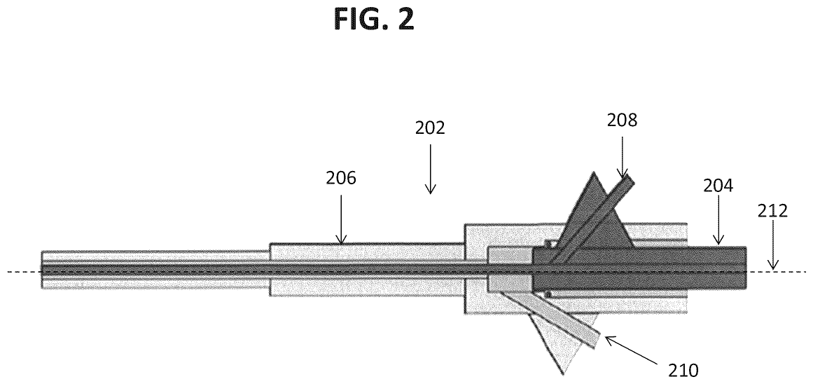

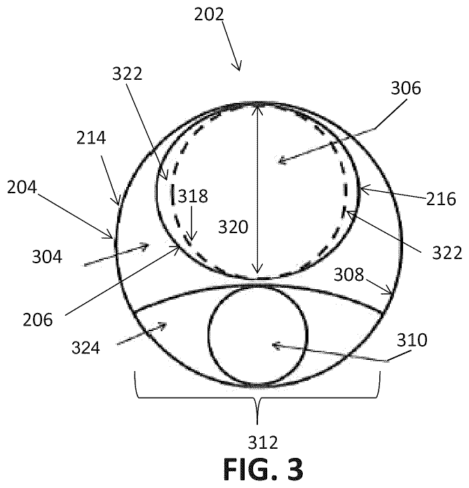

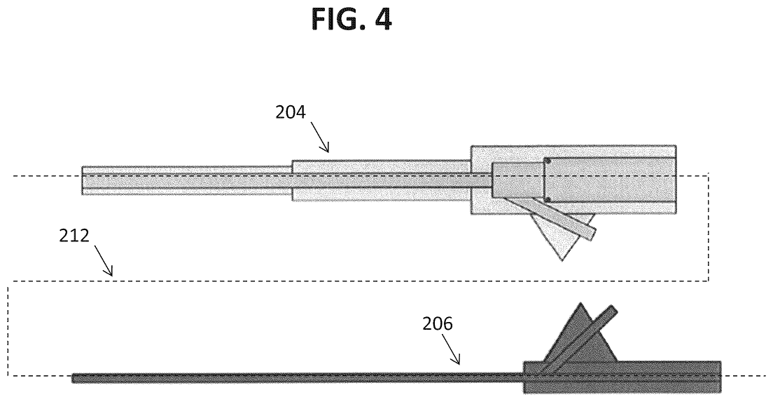

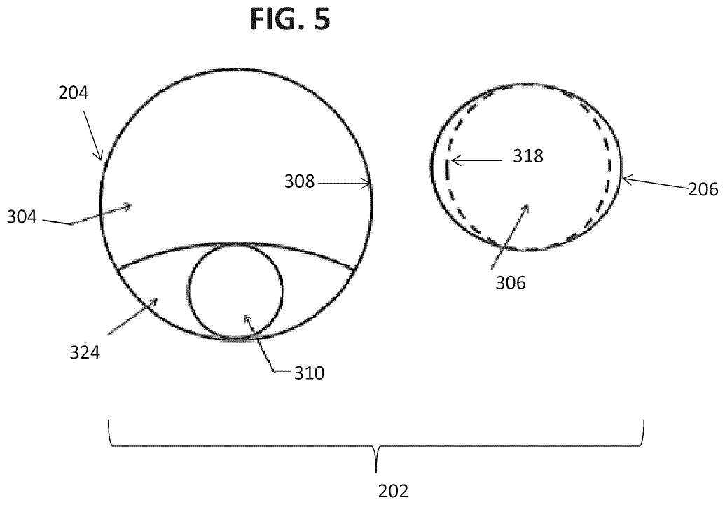

FIGS. 2-5 illustrate another continuous flow endoscope 202 provided in accordance with the present disclosure. Continuous flow endoscope 202 is shown in an assembled condition in FIGS. 2 and 3 and in a disassembled condition in FIGS. 4 and 5. Except as specifically contradicted below, continuous flow endoscope 202 may be configured similarly as continuous flow endoscope 100 (FIGS. 1A and 1B), may include any of the features thereof, and/or may form a system similarly as continuous flow endoscope 100 (FIGS. 1A and 1B), e.g., in conjunction with pump “P” and/or an outflow reservoir “0” (see FIG. 1A). In embodiments, continuous flow endoscope 202 differs from continuous flow endoscope 100 (FIGS. 1A and 1B) in that continuous flow endoscope 202 is configured for disassembly (into first and second subassemblies 204, 206) and in the arrangement of the internal features thereof, as detailed below.

Continuous flow endoscope device 202 includes a first subassembly 204 and a second subassembly 206 telescopically insertable into and removable from first assembly 204 along a shared central axis 212. First subassembly 204 includes an elongated tubular body having a first channel 304 and a plurality of optical components 310 disposed within an interior thereof, and may contain a fluid port 208. Second subassembly 206 includes an elongated tubular body having a second channel 306 of continuous flow endoscope device 202 disposed within an interior thereof and may contain one or more fluid ports 210. Alternatively, second subassembly 206 may including both fluid ports 208, 210.

Continuous flow endoscope device 202 allows for a variety of geometries, including those with corner-like or sharp transitions in channel cross-sections, to be employed, since first subassembly 204 and second subassembly 206 may be disassembled to facilitate cleaning and sterilization. Continuous flow endoscope device 202 is operational when the subassemblies 204, 206 are assembled, but not when they are disassembled. When continuous flow endoscope device 202 is in the assembled condition, a plurality of mating features (not explicitly shown) on first and second subassemblies 204, 206, respectively, are configured to lock second subassembly 206 relative to first subassembly 204. In some embodiments, the mating features (not explicitly shown) may include a single-button (single-digit) release mechanism that may include spring latch functionality. In addition, lead-in features (not explicitly shown) on the first and second subassemblies 204, 206, respectively, may be provided to align and guide the second subassembly 206 into the first subassembly 204 to prevent wedging or jamming during assembly.

First channel 304 of first subassembly 204 occupies an area within the interior of the elongated tubular member of first subassembly 204. More specifically, first channel 304 occupies a space which may be defined by inner surface 308 of the elongated tubular member of first subassembly 204 and is fluidly isolated from the plurality of optics components 310. Optics components 310 are located within an oculiform region 312 of the elongated tubular member of first subassembly 204 between a portion of inner surface 308 of the elongated tubular member of first subassembly 204 and an interior wall that fluidly isolates oculiform region 312 from first channel 304. First channel 304 may be used for fluid inflow. A second channel 306 disposed within the elongated tubular member of second subassembly 206 may be configured to accommodate a wide range of instruments 318, and may be used for fluid outflow. When second subassembly 206 is disposed within the elongated tubular member of first subassembly 204, the second channel 306 of second subassembly 206 is thus likewise disposed within and occupies a portion of the interior of the elongated tubular member of first subassembly 204. In some embodiments, a throttle feature (not shown) is disposed within the fluid outflow path to increase fluid resistance when an instrument 318 is not disposed within the second channel 306.

Optical components 310 are constrained within an oculiform region 312 at the bottom of the cross-section of the elongated tubular member of first subassembly 204. Optical components 310 may include but are not limited to a rod lens system, coherent fiber optic bundle, miniature camera components, illumination fibers, or LED light components. In an embodiment, a plurality of illumination fibers (light) 324 are located on either side of the optical components 310 within oculiform region 312. First channel 304 occupies the remaining space within the boundary of the cross-section of the elongated tubular member of first subassembly 204, resulting in a moon-shaped first channel 304.

In an embodiment, second channel 306 disposed within the elongated tubular member of second subassembly 206 may be defined at least in part by an elliptical cross-section of the elongated tubular member thereof, with a minor diameter 320 such that second subassembly 206 fits within first channel 304 of first subassembly 204 when the minor diameter 320 is oriented to extend between oculiform region 312 and an opposed portion of inner surface 308 of first subassembly 204. The minor diameter 320 of second channel 306 is such that an instrument 318 may be inserted through second channel 306, and, when a circular instrument is inserted, the instrument 318 may fully occupy the minor diameter 320 of second channel 306. It is appreciated that, in other embodiments, instruments defined in whole or in part by a cross-section or cross-sections that are not circular may also fully occupy the minor inner diameter 320. In an embodiment, additional space 322 remains within the second channel 306 on either side of the instrument 318, e.g., along the major diameter thereof, to allow for fluid outflow, e.g., true continuous flow, for the duration of the procedure. In an embodiment, when instrument 318 is removed from the second channel 306, continuous flow is maintained even if another instrument is not inserted in the second channel 306.

Continuous flow endoscope device 202 achieves continuous flow both with and without an instrument 318 therein and maximizes use of space within the endoscope cross-section. In particular, use of space is maximized because the optical components 310, channels 304, 306, and structural elements 204, 206 occupy 100% of the cross-sectional area of the working section, e.g., the interior area defined by the elongated tubular member of the first subassembly 204. This space-use efficiency allows for equivalent or better fluid performance at a reduced outer dimension for less traumatic access. In addition, the fluid resistance to inflow does not measurably vary during a procedure, allowing for better cavity pressure control when used with a fluid pump system, and disassembly allows for improved ability for cleaning and sterilization, thereby enhancing patient safety.

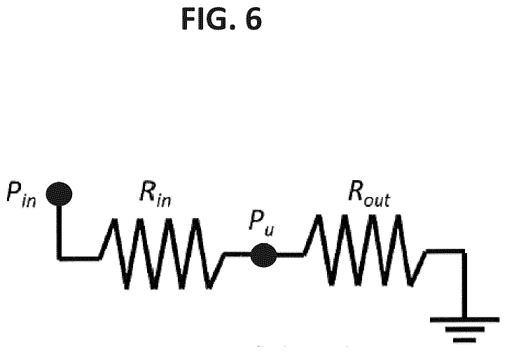

FIG. 6 represents a fluid circuit of an endoscope modeled on a first-order basis as two resistors in series. The first resistor, Rin, is the fluid impedance of the inflow channel, and the second resistor Rout is the fluid impedance of the outflow channel. The point between the resistors Rin and Rout represents the pressure within the cavity or organ Pu. In a steady flow state, the cavity pressure Pu remains constant, and the flow rates Rin and Rout are equal. Pressure supplied to the scope at the inflow Rin is indicated as Pin and the outflow pressure is assumed to be zero, or ground.

The ability of fluid to clear the visual field may be dependent upon the fluid flow rate in and out of the cavity, which is equal in a steady flow state. See Equation (1):

$$\\ \begin{matrix}Q = \frac{{P}_{in}}{( {R}_{in} + {R}_{out} )} & ( 1 )\end{matrix}$$

The flow rate depends upon the fluid impedances of both the inflow and outflow channels; an increase will result in a decrease in flow rate and subsequently a decrease in ability to clear the visual field. As such, general cross-sectional areas of both the inflow and outflow may be increased (to increase their combined resistance) in order to achieve sufficient (usable) overall flow rate.

The ability to maintain distension of the organ or cavity depends on the pressure in the cavity. In the series-resistor model, with the outflow connected to ground (as opposed to vacuum), the overall pressure difference across the scope is equal to the inflow pressure supplied to the scope, given in Equation (2):

Pᵢₙ=Q·(Rᵢₙ+Rₒᵤₜ) (2)

The cavity pressure is given in Equation (3):

Pᵤ=Q·(Rₒᵤₜ) (3)

Substitution results in the Equation (4), an expression for cavity pressure as a function of inflow pressure and fluid channel resistances:

$$\\ \begin{matrix}{P}_{u} = {P}_{in} ( \frac{{R}_{out}}{{R}_{in} + {R}_{out}} ) & ( 4 )\end{matrix}$$

Theoretically, it is always possible to increase the inflow pressure in order to increase the cavity pressure. However, there are practical limits on the inflow pressure: a fluid bag may only be hung so high in an operating room and fluid pumps have safety limits on inflow pressure. As such, it is desirable to maintain the cavity pressure as close as possible to the inflow pressure in order to provide sufficient distension. This is accomplished by maximizing the resistance of the outflow with respect to the resistance of the inflow, as provided in Equation (5):

$$\\ \begin{matrix}{lim}_{\frac{{R}_{{out}_{}}}{{R}_{in}} \to ∞} {P}_{u} = {lim}_{\frac{{R}_{{out}_{}}}{{R}_{in}} \to ∞} {P}_{in} ( \frac{{R}_{out}}{{R}_{in} + {R}_{out}} ) = {P}_{in} & ( 5 )\end{matrix}$$

As such, in order to increase the fluid performance of the endoscope: the sum of the fluid resistances of the inflow and outflow should be decreased to increase fluid flow rate (ability to clear visual field); the ratio of the outflow fluid resistance to the inflow fluid resistance should be increased to keep the cavity pressure as close as possible to the inflow pressure (maintain distension); and any instruments inserted through the endoscope which provide an additional outflow path should also be considered.

Decreasing fluid resistance may be achieved by increasing the cross-sectional area available for fluid flow. As such, a primary challenge in continuous flow endoscope design is to provide sufficient cross-sectional area for fluid flow within an endoscope outer diameter that is small enough to allow atraumatic access to the operative field. The above-detailed embodiments overcome these challenges.

While exemplary embodiments have been shown and described, modifications thereof can be made by one skilled in the art without departing from the scope or teachings herein. The embodiments described herein are exemplary only and are not limiting. Many variations and modifications are possible and are within the scope of the present disclosure. Accordingly, the scope of protection is not limited to the exemplary embodiments described herein, but is only limited by the claims that follow, the scope of which shall include all equivalents of the subject matter of the claims. Unless expressly stated otherwise, the steps in a method claim may be performed in any order.

1-20. (canceled)

21. A continuous flow endoscope, comprising:
an elongated tubular member having a longitudinal axis and defining a lumen in an interior of the elongated tubular member;
a first channel disposed within the lumen of the elongated tubular member, the first channel defining an interior space having a first cross-sectional area substantially perpendicular to the longitudinal axis, wherein a first lumen is defined through the interior space of the first channel;
a second channel disposed within the interior space of the first channel and defining a second lumen, the second channel configured to receive an instrument having a circular cross-section through the second lumen, the second lumen fluidly isolated from the first lumen, the second lumen defining a second cross-sectional area substantially perpendicular to the longitudinal axis, wherein the second cross-sectional area of the second lumen is different from the first cross-sectional area of the first lumen, and wherein the second cross-sectional area is non-circular such that, with the instrument inserted through the second lumen, additional space not occupied by the instrument is defined within the second lumen about at least a portion of the instrument; and
an optics device disposed in a free space of the lumen of the elongated tubular member, the free space being a space within the lumen of the elongated tubular member not occupied by the first channel,
wherein the first channel is arranged to pass fluid in a first direction and the second channel is arranged, with the instrument inserted through the second lumen, to pass fluid through the additional space in a second direction different from the first direction, the first and second channels arranged to isolate the fluid from the free space of the lumen of the elongated tubular member.

22. The continuous flow endoscope according to claim 21, wherein the first channel is dedicated to fluid inflow and the second channel is dedicated to fluid outflow.

23. The continuous flow endoscope according to claim 21, wherein the first channel is dedicated to fluid outflow and the second channel is dedicated to fluid inflow.

24. The continuous flow endoscope according to claim 21, wherein the second cross-sectional area defines an elliptical configuration having a major diameter and a minor diameter such that the additional space is defined along the major diameter on either side of the instrument when the instrument is inserted through the second lumen.

25. The continuous flow endoscope according to claim 21, wherein the second channel is further configured to operate without the instrument inserted through the second lumen.

26. The continuous flow endoscope according to claim 25, wherein the first and second channels are configured to maintain continuous fluid flow both with and without the instrument inserted through the second lumen.

27. The continuous flow endoscope according to claim 21, wherein the second channel is removable from the interior of the elongated tubular member.

28. The continuous flow endoscope according to claim 21, wherein the optics device is configured to be removably received in the free space of the interior of the elongated tubular member.

29. The continuous flow endoscope according to claim 21, wherein a shape of the second cross-sectional area is different from a shape of the first cross-sectional area.

30. The continuous flow endoscope according to claim 21, wherein the instrument is configured to be removably received in the second channel.

31. The continuous flow endoscope according to claim 21, wherein the instrument substantially fills the second lumen in at least one cross-sectional dimension of the second channel when the instrument is inserted through the second lumen.

32. The continuous flow endoscope according to claim 21, further comprising a plurality of illumination fibers disposed in the free space of the lumen of the elongated tubular member and at least partially surrounding the optics device.

33. The continuous flow endoscope according to claim 32, wherein the plurality of illumination fibers and the optics device fully occupy the free space of the lumen of the elongated tubular member.

34. A continuous flow endoscope, comprising:
a proximal body including an inflow and an outflow, the proximal body adapted to connect to an imaging device;
an elongated tubular member extending distally from the proximal body, the elongated tubular member defining a lumen having a circular cross-section, the lumen divided into first and second portions that are fluidly-isolated from one another, the second portion having an oculiform cross-section and the first portion defining a first channel having a cross-section of a circle with an oculiform cut-out;
a second channel disposed within first channel and defining a lumen having an elliptical cross-section, the second channel configured to receive an instrument through the lumen of the second channel such that, with the instrument inserted through the lumen of the second channel, additional space not occupied by the instrument is defined within the lumen of the second channel about at least a portion of the instrument; and
an optics device disposed in the second portion of the elongated tubular member,
wherein the first channel is fluidly coupled to one of the inflow or outflow and arranged to pass fluid in a first direction, and
wherein the second channel is fluidly coupled to another of the inflow or outflow and arranged to pass fluid in a second direction different from the first direction.

35. The continuous flow endoscope according to claim 34, wherein the elliptical cross-section of the lumen of the second channel has a major diameter and a minor diameter and wherein the instrument substantially fills the minor diameter of the lumen of the second channel when the instrument is inserted through the lumen of the second channel.

36. The continuous flow endoscope according to claim 34, further comprising a plurality of illumination fibers disposed in the second portion of the lumen of the elongated tubular member and at least partially surrounding the optics device.

37. The continuous flow endoscope according to claim 36, wherein the plurality of illumination fibers and the optics device fully occupy the second portion of the lumen of the elongated tubular member.

38. The continuous flow endoscope according to claim 34, wherein the first and second portions fully occupy the lumen of the elongated tubular member.
